(19) United States
(12) Patent Application Publication
(10) Pub. No.: US 2023/0225983 A1
(43) Pub. Date: Jul. 20, 2023

(54) DRUG COMPRISING PLGA NANOPARTICLES LOADED WITH CAPE TARGETED WITH ANGIOPEP-2 PEPTIDE

(71) Applicant: YILDIZ TEKNIK UNIVERSITESI, Istanbul (TR)

(72) Inventors: Serap DERMAN, Istanbul (TR); Banu MANSUROGLU, Istanbul (TR); Yagmur BOZKURT, Istanbul (TR); Tayfun ACAR, Istanbul (TR); Fatma SAYAN POYRAZ, Istanbul (TR)

(73) Assignee: YILDIZ TEKNIK UNIVERSITESI, Istanbul (TR)

(21) Appl. No.: 18/011,215

(22) PCT Filed: Dec. 7, 2020
(86) PCT No.: PCT/IB2020/001053

§ 371 Date: Dec. 19, 2022

(30) Foreign Application Priority Data
Jun. 18, 2020 (TR) 2020/09490

(51) Int. Cl. A61K 9/51; A61K 31/216; A61K 47/42

(52) U.S. Cl. CPC A61K 9/5153; A61K 31/216; A61K 47/42

(57) ABSTRACT

A cancer drug for use in the treatment of cancer in which caffeic acid phenethyl ester (CAPE) is encapsulated in poly [lactic-co-glycolic acid] (PLGA) to increase its solubility in aqueous media is provided. Angiopep-2 is used as a specific ligand to increase biological activity. In an embodiment of the invention, PLGA is preferably in the form of nanoparticles, thereby ensuring better solubility.

### CROSS REFERENCE TO THE RELATED APPLICATIONS

This application is the national phase entry of International Application No. PCT/IB2020/001053 filed on Dec. 7, 2020, which is based upon and claims priority to Turkish Patent Application No. 2020/09490 filed on Jun. 18, 2020, the entire contents of which are incorporated herein by reference.

### TECHNICAL FIELD

The present invention relates to a targeted drug with high water solubility. In particular, the present invention relates to a drug containing CAPE loaded PLGA nanoparticles targeted with the angiopep-2 peptide.

### BACKGROUND

PLGA (poly [lactic-co-glycolic acid]) is a polymer that is widely used in the medical field due to its biocompatibility, durability in biomedical applications and ease of administration through parenteral injection. Nanoparticles (NP's) prepared using the PLGA polymer is a FDA-approved (US Food and Drug Administration) drug delivery system which has higher stability and drug loading capacity compared to other nanocarriers, which is capable of delivering a sustained release, non-immunogenic, which reduces the toxicity of the drug, and increases the bioavailability and action/action time of the drug. In the in vitro and in vivo studies in the literature, it has been stated that PLGA NPs can cross the blood brain barrier by virtue of the surface modifications and the presence of targeting agents. In addition, when the free drug and the PLGA NP drug delivery systems are compared, it is known that the PLGA NP drug delivery systems are more effective and are among the components with the highest rate in the literature in terms of their ability to cross the blood brain barrier and penetrate the brain tissues.

Angiopep-2 peptide is a synthetic peptide that interacts with LRP-1 cell receptors, discovered by bioinformatic analysis of human proteins having the Kunitz sequence (TFFYGGSRGKRNFKTEEY-OH) and of the aprotinin sequences. The peptide, which has a highly hydrophilic structure, has a molecular weight of 2301.47 g/mol and an isoelectric point of pI9.91. Conjugated forms of drugs that have previously been successful in the blood brain barrier, such as doxorubicin [150], etoposide, and paclitaxel, with angiopep-2 peptide, have shown a greater success and proceeded to phases I and II in clinical trials for the treatment of glioblastoma. Angiopep-2 has been used to target small molecules, proteins and oligonucleotides to the central nervous system using several nanoparticular systems. In these studies, the ability of systems targeted with angiopep-2 to cross the blood brain barrier was increased by 1.5-3. Although small molecules and nanocarriers have been mostly studied with the angiopep-2 peptide, it has been reported that it is also highly effective in the delivery of antibodies.

Caffeic acid phenethyl ester (CAPE) is one of the main components of propolis produced by honeybees. CAPE has many important biological activities including anti-bacterial, antiviral, antifungal, antioxidant, anti-inflammatory, and anti-cancer properties. It has been shown that CAPE is cytotoxic to many types of cancer cells, including breast cancer cells, but has no such effect against normal cells.

The use of the biocompatible CAPE molecule, whose cytotoxic effect on cancer cells is already known, as an active ingredient is extremely limited due to the low water solubility of the molecule. The low solubility of the drug affects its pharmacodynamics in living systems, thereby reducing the effectiveness of the drug as well as the treatment. Furthermore, the administration of the drug in free form leads to a decrease in the amount of the molecule reaching the diseased area resulting from its random distribution in the circulatory system. This leads to an increase in the amount and frequency of the dose to be administered and causes a decrease in the pharmacokinetic effectiveness of the drug. In cytotoxicity studies on cancer cells with the free form of the CAPE molecule, IC₅₀ (minimum inhibition concentration) concentrations are very high due to the low solubility of the drug and its inability to interact through cell-specific receptors.

In an article by Derman (Journal of Nanomaterials, vol. 16, p. 318, 2015), the delivery system is PLGA nanoparticles. The solubility in water is higher than the free form. With regard to the cell/tissue-specific targeting, biological activity analysis has not been conducted, and the problem remains to be solved.

In a study by Arasoglu et al. (Nanotechnology, vol. 27, p. 025103, 2015), the delivery system is PLGA nanoparticles. The solubility in water is higher than the free form. With regard to the cell/tissue-specific targeting, only antimicrobial studies have been conducted, but no solution has been provided to the problem.

In a study by Abamor (Asian Pacific journal of tropical medicine, vol. 10, pp. 25-34, 2017), the delivery system is PLGA nanoparticles. The solubility in water is higher than the free form. With regard to the cell/tissue-specific targeting, only antileishmanial activity was evaluated, but no solution has been provided to the problem.

In a study by Saelo et al. (Journal of agricultural and food chemistry, vol. 64, pp. 6694-6707, 2016), the delivery system is nanoparticle-based cellulose films. Nanoparticles agglomerated after storage, and accordingly their solubility in water decreased. Accordingly, the water solubility of the CAPE molecule has also inevitably decreased along with the delivery system. With regard to the cell/tissue-specific targeting, although no targeting ligand is used, it was determined by a DLS analysis that nanoparticles of large sizes (50-625 nm) were obtained, which does not allow a possible active targeting. Antibacterial activity has been studied in the article in question.

In a study by Arasoglu and Derman (Journal of agricultural and food chemistry, vol. 66, pp. 6196-6204, 2018), the delivery system is PLGA nanoparticles. The solubility in water is higher than the free form. No solution has been provided for cell/tissue-specific targeting.

In a study by Garrido et al. (Food chemistry, vol. 254, pp. 260-265, 2018), the delivery system is hydroxypropyl β cyclodextrin complexes. The amount of the CAPE molecule used (20 mg) is quite low. No solution has been provided regarding the decrease in the water solubility of CAPE in high amounts. Although the problem of solubility in water has been studied, no solution has been provided for cell/tissue-specific targeting.

In a study by Guan et al. (Journal of food engineering, vol. 246, pp. 125-133, 2019), the delivery system is sucrose fatty acid ester nanoparticles. No solution has been provided for water solubility. Although the cytotoxic activity of the nanoparticles produced has been studied, no solution has been provided for cell/tissue-specific targeting.

In a study by Tambuwala et al. (Drug delivery and translational research, vol. 9, pp. 14-24, 2019), the delivery system is albumin nanoparticles. The amount of the CAPE molecule used (30 mg) is quite low. No solution has been provided regarding the decrease in the water solubility of CAPE in high amounts. Particle sizes are quite large for possible active targeting studies. Therefore, no solution has been provided for cell/tissue-specific targeting.

In a study by Yoncheva et al. (Biotechnology& Biotechnological Equipment, vol. 33, pp. 64-74, 2019), PEO-b-PCL-b-PEO micelles were used as the delivery system. The amount of the CAPE molecule used (2 mg) is quite low. No solution has been provided regarding the decrease in the water solubility of CAPE in high amounts. No solution has been provided for cell/tissue-specific targeting.

In a study by Lee et al. (Journal of pharmaceutical sciences, vol. 104, pp. 144-154, 2015), the delivery system is PEG-PCL nanoparticles. The amount of the CAPE molecule used (5-10 mg) is quite low. No solution has been provided regarding the decrease in the water solubility of CAPE in high amounts. No solution has been provided for cell/tissue-specific targeting.

In a study by Gupta et al. (Journal of Photochemistry and Photobiology B: Biology, vol. 174, pp. 235-242, 2017), MnO₂/NiO nanocomposites were used as the delivery system. The water solubility of drugs is higher in nanoparticular systems; however, polymeric systems are more advantageous than metallic systems. No solution has been provided for cell/tissue-specific targeting.

In the International Patent Application Publication No. WO 2019/132831 A2, tissue-derived exosomes were used as the delivery system and the solubility in water was increased. The exosomes used in the said publication naturally target the neuroblastoma cancer type. Therefore, the carriers used therein are non-synthetic polymer carriers, that are naturally sourced and protein in nature. Since the exosomes used as carriers originate from living tissues, they carry the risk of causing inflammation by triggering the immune system. This is one of the biggest obstacles to the use of such drug delivery systems in humans.

For the reasons above, there is still a need to develop a drug that has high solubility and offers effective treatment even at low doses.

The main object of the invention is to provide solutions to the problems in the prior art. Another object of the invention is to provide a cancer drug with high solubility, particularly a drug for glioma. Another object of the invention is to provide a cancer drug, particularly a drug for glioma, which has high pharmacokinetic activity even at low doses. Another object of the invention is to provide a cancer drug, particularly a drug for glioma, having improved water solubility and improved pharmacokinetic properties.

### SUMMARY

The present invention provides a cancer drug in which caffeic acid phenethyl ester (CAPE) is encapsulated in poly [lactic-co-glycolic acid] (PLGA), thereby improving its water solubility. The pharmacokinetic properties of the said drug are improved by using angiopep-2 as a targeter. PLGA is preferably in the form of nanoparticles; thus, by keeping the contact surface with the aqueous medium high per volume of each PLGA block, the solubility is further improved. Therefore, a drug is provided, which has high pharmacokinetic properties even at low doses, especially in the treatment of glioma.

### DETAILED DESCRIPTION OF THE EMBODIMENTS

In the state of the art, there is no publication about CAPE loaded PLGA nanoparticles directly targeted with angiopep-2; and studies investigating CAPE, PLGA and angiopep-2 peptide, individually, have already been listed. Various studies on the drug delivery systems in which is aimed to overcome the low water solubility problem of the CAPE molecule have been mentioned in the background art section above. Although the above-mentioned articles in the prior art provide a solution to water solubility, the amount of CAPE used is quite low and it has disadvantages against the problem of water solubility that is aggravated in increasing amounts. In the International Application Publication No. WO 2019/132831 A2, the CAPE molecule was targeted against neuroblastoma cancer cells using exosomes, and ligands were not used specifically for targeting. In the state of the art, there is no solution for improving the cytotoxic activity and yet targeting the molecule with cell-specific ligands.

In the present improvement, a PLGA-based polymeric carrier is used because of its biocompatible and biodegradable behavior. Targeting is provided by the angiopep-2 peptide. The drug delivery systems of the present application, targeted with cell-specific ligands, both increase the water solubility of the molecule and increase biological activity by triggering various receptor-mediated cell entry mechanisms.

The delivery system used in the present improvement is a synthetic polymer, and specifically only the blood brain barrier and glioma cells are targeted with the angiopep-2 peptide. Synthetic PLGA nanoparticles are approved by the FDA and are suitable for use in humans and other living organisms. PLGA is used for the specific delivery of the CAPE, and to increase its solubility in water. The CAPE molecule is encapsulated in PLGA polymeric nanoparticles, so that its water solubility is increased. Meanwhile, angiopep-2 peptide is attached to the surface of the nanoparticles, and the LRP-1 receptor located on the blood brain barrier endothelial cells and on the surface of glioma cells is targeted. Thus, selectivity in treatment is provided and the biological activity of the molecule is increased.

Thus, the present invention provides a drug containing PLGA with CAPE encapsulated therein, and angiopep-2 that is preferably attached to the surface thereof. CAPE is used as a cytotoxic agent, thus ensuring the biological activity of the drug delivery system obtained as a product. CAPE is provided encapsulated in PLGA.

In a preferred embodiment of the invention, Angiopep-2 is used in the product in order to target CAPE to the brain barrier and glioma cells, so that a high biological activity is ensured. Accordingly, a preferred embodiment of the present invention is a drug containing angiopep-2. Angiopep-2 may be located on the surface of the PLGA.

PLGA blocks are hydrophobic and may be produced in nanoparticular structure to confine CAPE. Thus, a preferred embodiment of the product of the present application is a drug containing PLGA nanoparticles with CAPE encapsulated therein. Preferably, angiopep-2 is located on the surface of the said PLGA nanoparticles.

In an exemplary embodiment of the invention, a drug formulation is provided that can cross the blood brain barrier, for the treatment of patients having tumors of the central nervous system or glioma brain tumors. CAPE loaded nanoparticles targeted with the angiopep-2 peptide can bind to the LRP-1 receptors in the blood-brain barrier endothelial cells by means of the said peptide and pass into the central nervous system via transcytosis (i.e., receptor-mediated transcytosis). Thanks to the effect of the CAPE molecule in the treatment of the central nervous system diseases and glioma, the treatment may be performed with high efficiency. With the targeting angiopep-2 peptide, glioma cells may also be specifically targeted, thereby making it possible to achieve selectivity in treatment and to cross the blood-brain barrier.

The water solubility of the CAPE molecule with known biocompatibility is increased by present improvement. The drug provided may also be used as an interim treatment drug that will not cause any side effects between chemotherapy sessions, as it provides cell/tissue-specific targeting.

It has been concluded that the cytotoxic activity of the product synthesized and characterized in laboratory studies on glioma cells is higher than the free CAPE.

With the improvement of the invention, the following objectives have been achieved:
the deficiencies in the state of the art have been eliminated, problems have been solved;
a cancer drug with high solubility, particularly a drug for glioma, is provided;
a high pharmacokinetic activity of the said drug is also obtained.

What is claimed is:

1. A drug for a use in treating a cancer, comprising caffeic acid phenethyl ester (CAPE) encapsulated in poly [lactic-co-glycolic acid] (PLGA).

2. The drug according to claim 1, further comprising angiopep-2 attached to a surface of the PLGA, as a targeter.

3. The drug according to any one of claim 1 , wherein the PLGA is in a form of nanoparticles.

4. The drug according to any one of claim 1, wherein the drug is used to treat glioma.

5. The drug according to claim 2, wherein the PLGA is in a form of nanoparticles.

6. The drug according to claim 2, wherein the drug is used to treat glioma.

7. The drug according to claim 3, wherein the drug is used to treat glioma.
